(19) United States
(12) Patent Application Publication
(10) Pub. No.: US 2019/0355453 A1
(43) Pub. Date: Nov. 21, 2019

(54) Method of Universal Medical Information Distribution

(71) Applicant: Anna Lynn Feigenwinter, Clifton Park, NY (US)

(72) Inventor: Anna Lynn Feigenwinter, Clifton Park, NY (US)

(21) Appl. No.: 16/418,431

(22) Filed: May 21, 2019

Related U.S. Application Data
(60) Provisional application No. 62/674,096, filed on May 21, 2018.

(51) Int. Cl. G16H 10/60; H04L 29/06; G16H 50/20

(52) U.S. Cl. CPC G16H 10/60; G16H 50/20; H04L 63/102

(57) ABSTRACT

A system and method of universal medical information distribution. The method includes at least one medical professional account, at least one patient account, and a plurality of medications managed by the remote server. The method provides the most current and accurate medical history of a patient account for at least one medical professional. The method begins with prompting the medical professional account to upload a patient history through a medical professional portable computing (PC) device. A patient profile is generated for the patient account. The medical professional account is prompted to upload a new medication request through the medical professional PC device. The new medication request is compared to the at least one conflicting medication in order to identify at least one adverse medication interaction match. An adverse medication notification is displayed with the medical professional PC device if the at least one adverse medication interaction is identified.

The current application claims priority to U.S. provisional application Ser. No. 62/674,096 filed on May 21, 2018.

### FIELD OF THE INVENTION

The present invention generally relates to distribution of medical information. More specifically, the present invention is a method of universal medical information distribution.

### BACKGROUND OF THE INVENTION

There have been numerous advancements in medical treatment. From heart transplants to drugs that cure infections, the field of patient continues to develop advanced techniques for improving medical outcomes. While the drugs being developed are able to provide targeted solutions to diverse problems, there are often negative interactions between various drugs. Therefore, it is more important than ever to make sure that medical professionals are able to view all of the drugs a patient is receiving.

The present invention addresses this need by providing a method for sharing medical information of a patient with a group of authorized medical professionals. This enables a patient to receive the best possible care. Additionally, the method of the present invention is designed to work with a medical information device that stores all of the medical information for the patient. As a result, medical professionals are able to view all of the medical information of the patient with or without access to a remote datacenter. The medical information device facilitates the achievement of positive patient outcomes by acting as an on-demand medical records file.

Furthermore, a patient identifier device can be used as a form of medical identification for patients that visit a hospital. For example, the patient identifier device is preferably a card with an integrated memory chip that an individual carry with them. If the individual were unable to respond to emergency response team members, an emergency response member would be able to quickly look for the card, engage the patient identifier device with a reader, and access the medical history and drug information of the individual. This functionality is achieved by leveraging the interoperability of data exchange between medical facilities, to enable universal access to the medical records of an individual.

### BRIEF DESCRIPTION OF THE DRAWINGS

FIG. 1 is a diagram illustrating electronic interactions in the present invention.

FIG. 2 is a flowchart illustrating the overall process for the present invention.

FIG. 3 is a flowchart illustrating the process to grant access to a medical professional account to view a patient profile with a medical professional PC device for the present invention.

FIG. 4 is a flowchart illustrating the process to append the new set of credentials with the medical professional account with the remote server for the present invention.

FIG. 5 is a flowchart illustrating the process to display a condition conflict message through the medical professional PC device if at least one adverse medication interaction is identified for the present invention.

FIG. 6 is a flowchart illustrating the process to display a condition conflict message through the medical professional PC device if a harmful medication is identified for the present invention.

FIG. 7 is a flowchart illustrating the process to generate a prescription notification from the new medication request for the present invention.

FIG. 8 is a flowchart illustrating the process to append the new medication request into the at least one taken medication for the patient profile for the present invention.

FIG. 9 is a flowchart illustrating the process to compare the new set of credentials with a set of verified credentials for the present invention.

FIG. 10 is a flowchart illustrating the process to send a prescription notification to a pharmaceutical PC device for the present invention.

FIG. 11 is a flowchart illustrating the process to display a condition conflict message through the pharmaceutical professional PC device if a harmful medication is identified for the present invention.

FIG. 12 is a flowchart illustrating the process to display a condition conflict message through the pharmaceutical professional PC device if the at least one adverse medication interaction is identified for the present invention.

FIG. 13 is a flowchart illustrating the process to alert the pharmaceutical account of the modification through the pharmaceutical PC device for the present invention.

FIG. 14 is a flowchart illustrating the process to display a confirmation through the medical professional PC device for the present invention.

FIG. 15 is a flowchart illustrating the process to prompt a supplementary medical professional account to send a confirmation to the primary medical professional account through a supplementary medical professional PC device for the present invention.

FIG. 16 is a flowchart illustrating the process to display the confirmation through a primary medical professional PC device for the present invention.

### DETAIL DESCRIPTIONS OF THE INVENTION

All illustrations of the drawings are for the purpose of describing selected versions of the present invention and are not intended to limit the scope of the present invention.

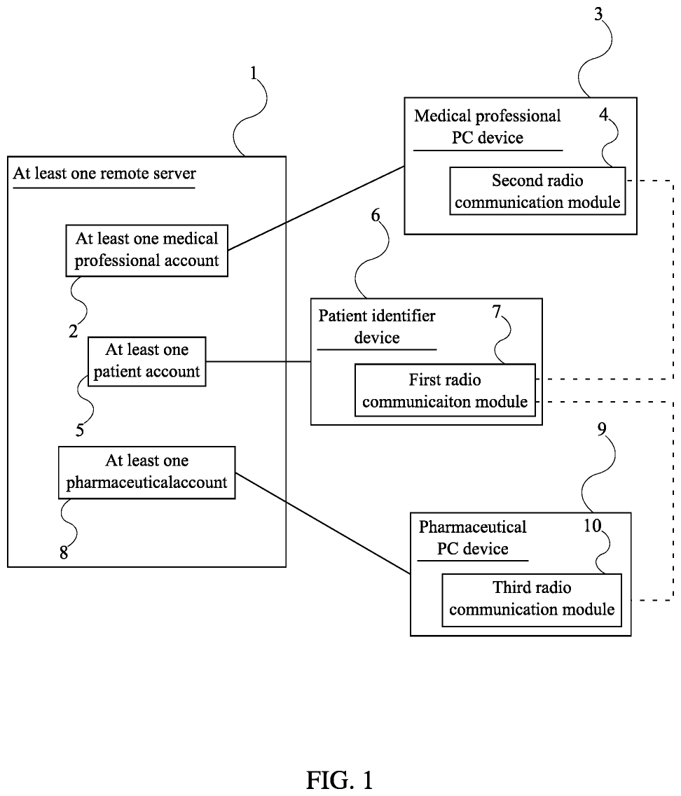

The present invention is a method of universal medical information distribution. The present invention provides all medical history of a patient to be accessed by an authorized medical professional. More specifically, the present invention prevents the combination of harmful or lethal medications based on any current medical condition, patient history, and any medications a patient may be currently taking. The present invention may be utilized in everyday medical appointments as well as emergencies when a patient or a point of contact is not able to provide medical information regarding the patient. Thus, as seen in FIG. 1, the physical system used to implement the method of the present invention includes at least one remote server 1, a medical professional personal computing (PC) device 3, and a patient identifier device 6. At least one medical professional account 2 is managed by at least one remote server 1, wherein the at least one medical professional account 2 is associated to a medical professional PC device 3 (Step A). All data associated with the at least one medical professional account 2 is stored on the at least one remote server 1, and any inputs through the medical professional PC device 3 is executed via the at least one remote server 1. Similarly, at least one patient account 5 and a plurality of medications are managed by the remote server 1, wherein each patient account 5 is associated with at least one taken medication from the plurality of medications. In order to notify a medical professional of an adverse reaction, each taken medication is associated to at least one conflicting medication from the plurality of medications. The at least one taken medication includes current medications and previously taken medications of the patient account 5. Moreover, each patient account 5 is stored on a patient identifier device 6 (Step B). The patient identifier device 6 is preferably a card that may be scanned or swiped with a compatible and authorized device, preferably the medical professional PC device 3. The patient identifier device 6 comprises the necessary electronic components that allow patient information to be accessible with the scan or swipe of the patient identifier device 6. Any data associated with that at least one patient account 5 is stored on the at least one remote server 1 through the patient identifier device 6. The patient identifier device 6 and the medical professional PC device 3 communicate with each other via the at least one remote server 1.

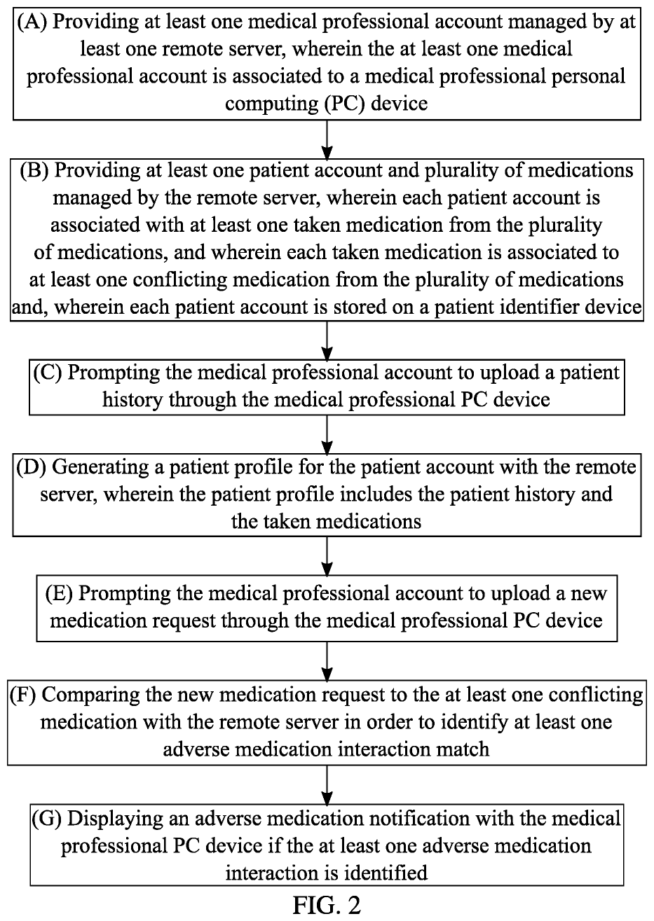

The overall process for the present invention includes the following steps that are implemented with the at least one remote server 1, the medical professional personal computing (PC) device 3, and the patient identifier device 6. As seen in FIG. 2, the overall process begins by prompting the medical professional account 2 to upload a patient history through the medical professional PC device 3 (Step C). The patient history preferably includes medical information requested by medical forms such as prior medical conditions, previous surgical operations, and so on. In order to identify each patient account 5 and the current medical status of each patient account 5, a patient profile is generated for the patient account 5 with the remote server 1, wherein the patient profile includes the patient history and the taken medications (Step D). Any harmful or fatal reactions are prevented during a medical visit as the medical professional account 2 is prompted to upload a new mediation request through the medical professional PC device 3 (Step E). The new medication request is compared to the at least one conflicting medication with the remote server 1 in order to identify at least one adverse medication interaction match (Step F), thereby automatically and accurately accounting for harmful and fatal reactions during the medical visit. The medical professional account 2 is alerted of a harmful or fatal reaction as an adverse medication notification is displayed with the medical professional PC device 3 if the at least one adverse medication interaction is identified (Step G).

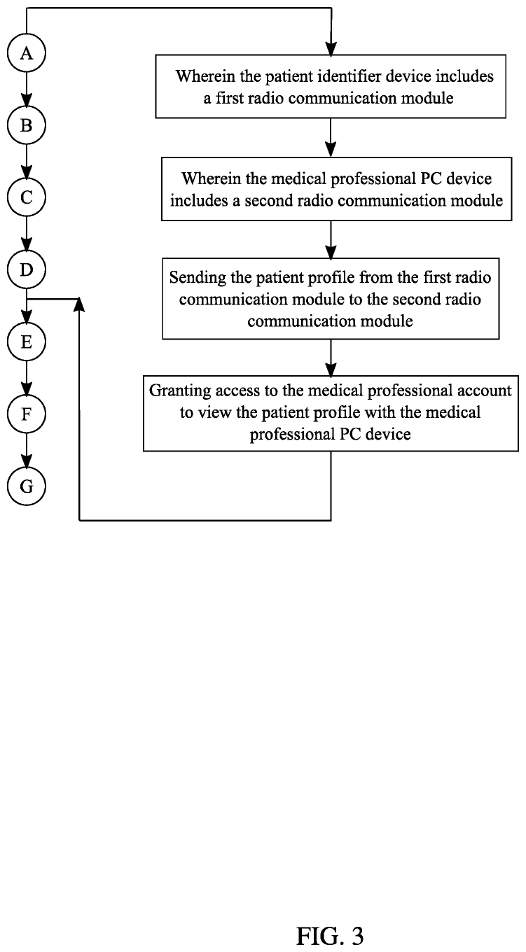

In order for the patient identifier device 6 to communicate with the medical professional PC device 3, the patient identifier device 6 includes a first radio communication module 7. Similarly, the medical professional PC device 3 includes a second radio communication module 4, seen in FIG. 3. The first radio communication module 7 and the second radio communication module 4 allow information to be shared and transferred between the patient identifier device 6 and the medical professional device. More specifically, the first radio communication module 7 and the second radio communication module 4 allow the patient identifier device 6 to be scanned with the medical professional PC device 3. With the patient identifier device 6 within a given distance of the medical professional PC device 3, the patient profile is sent from the first radio communication module 7 to the second radio communication module 4. Access is granted to the medical professional account 2 to view the patient profile with the medical professional PC device 3, therefore permitting access to a patient account 5 and the current medical status of the patient account 5 with the patient identifier device 6.

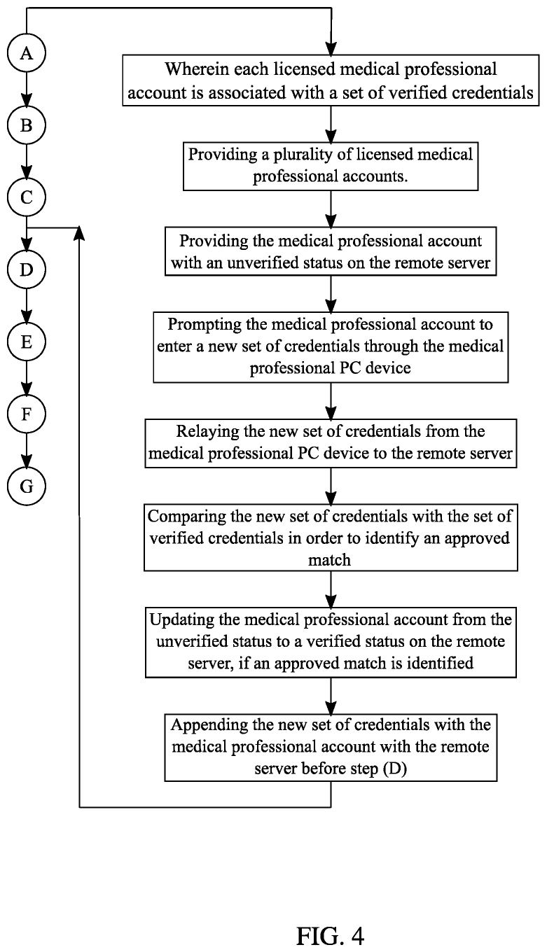

The medical information of a patient account 5 is protected by ensuring the medical professional who is able to access the patient account 5 is in fact a licensed medical professional. In order to confirm the status of a medical professional, a plurality of licensed medical professional accounts is provided, wherein each licensed medical professional account 2 is associated with a set of verified credentials, seen in FIG. 4. The medical professional account 2 is initially provided with an unverified status on the remote server 1. In order to identify the medical professional account 2 among other medical professional accounts, the medical professional account 2 is prompted to enter a new set of credentials through the medical professional PC device 3. The new set of credentials is relayed from the medical professional PC device 3 to the remote server 1 and is compared with the set of verified credentials in order to identify an approved match. The medical professional account 2 is updated from the unverified status to a verified status on the remote server 1, if an approved match is identified. The new set of credentials is appended with the medical professional account 2 with the remote server 1 before step (D), thereby allowing the medical professional to view and update a patient profile.

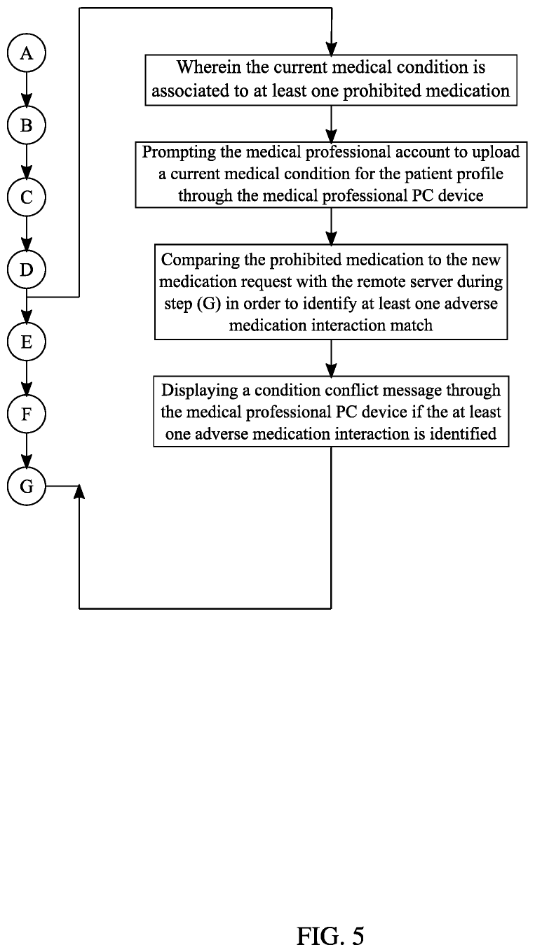

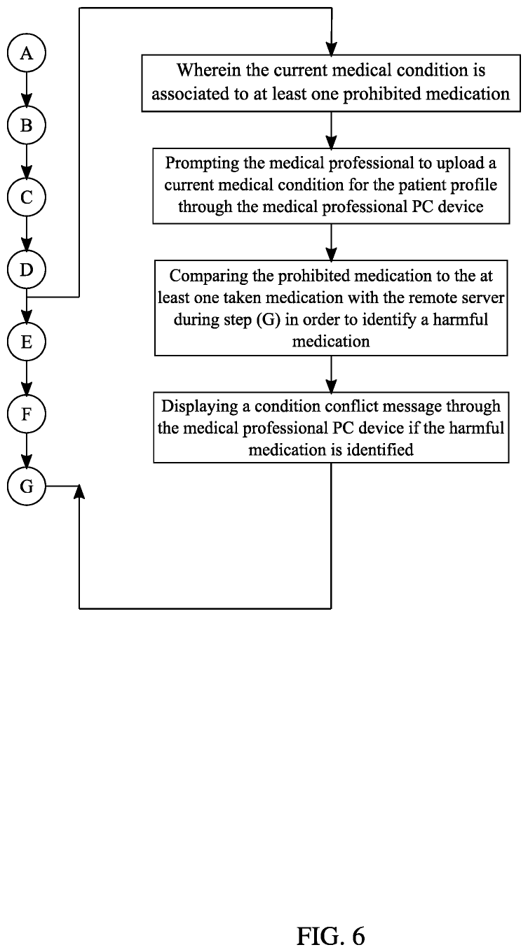

If a patient account 5 is experiencing a new medical condition, the present invention prevents any adverse interactions with new medications as the medical professional account 2 is prompted to upload a current medical condition for the patient profile through the medical professional PC device 3. The current medical condition is associated to at least one prohibited medication, seen in FIG. 5. The current medical condition may include an injury that requires an invasive procedure, a pregnancy, a new allergy, and so on. The prohibited medication is compared to the new medication request with the remote server 1 during step (G) in order to identify at least one adverse medication interaction match. More specifically, if any new medication is given or prescribed by the medical professional account 2, the harmful or fatal reaction with the patient account 5 is immediately made known to the medical professional account 2. The medical professional account 2 is made aware of the harmful or fatal reaction as a condition conflict message is displayed through the medical professional PC device 3 if the at least one adverse medication interaction is identified. The present invention further ensures the safety of the patient account 5 by alerting the medical professional account 2 of any harmful or fatal reactions of medications currently being taken by the patient account 5, seen in FIG. 6. Similar to any new medication given or prescribed by the medical professional, the prohibited medication is compared to the at least one taken medication with the remote server 1 during step (G) in order to identify a harmful medication. A harmful medication is accounts for taken medications that are prescribed more than once, by a medical professional account 2 or multiple medical professional accounts of the at least one medical professional account 2. More specifically, at least one conflicting medication of a taken medication preferably includes a duplicate prescription request of the corresponding taken medication of a patient profile for the patient account 5. A conflict message is displayed through the medical professional PC device 3 if the harmful medication is identified.

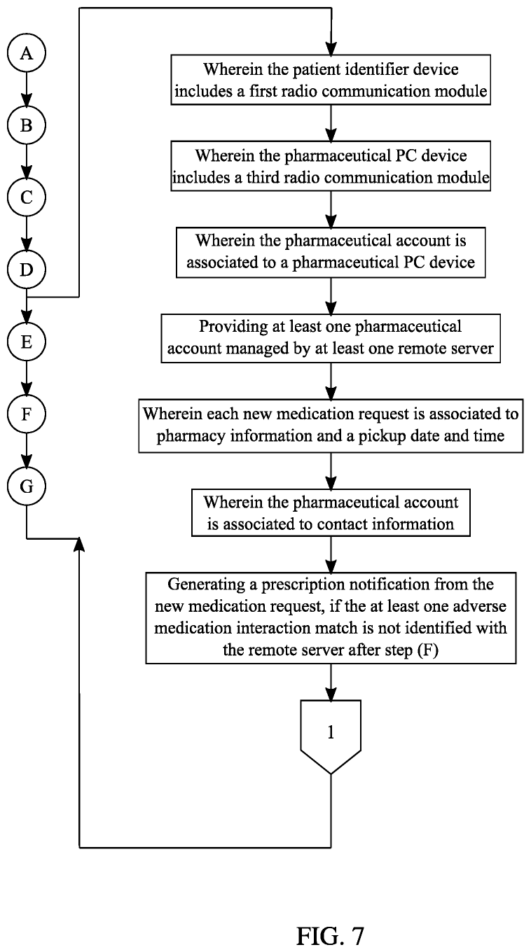

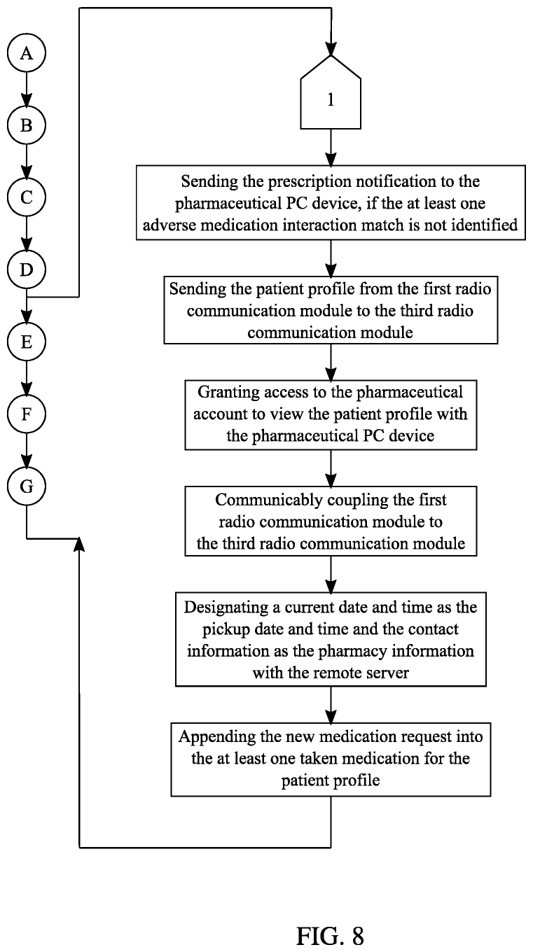

Once the medical professional account 2 has viewed a patient account 5, made any necessary updates to the patient profile, and a new medication prescribed by the medical professional has been approved by the present invention, a prescription may be requested by the medical professional account 2 for the patient account 5. As seen in FIG. 7 and FIG. 8, the present invention further ensures the safety of the patient account 5 by providing at least one pharmaceutical account 8 that is managed by the at least one remote server 1, wherein the pharmaceutical account 8 is associated to a pharmaceutical PC device 9. Providing a pharmaceutical account 8 enhances the accountability of the present invention such that all medical providers are verified and archived. In order for the pharmaceutical account 8 to process a prescription from the new medication request, a prescription notification is generated from the new medication request, if the at least one adverse medication interaction match is not identified with the remote server 1 after step (F). The prescription notification is sent to the pharmaceutical PC device 9, if the at least one adverse medication interaction match is not identified. Moreover, all prescriptions requested by a medical professional with the present invention are safe for the patient account 5.

Similar with the medical professional account 2, the pharmaceutical account 8 is able to access a patient account 5 with patient identifier device 6, also seen in FIG. 7 and FIG. 8. In order for the pharmaceutical PC device 9 to communicate with the patient identifier device 6, the pharmaceutical PC device 9 includes a third radio communication module 10. The patient profile is sent from the first radio communication module 7 to the third radio communication module 10, and the pharmaceutical account 8 is granted access to view the patient profile with the pharmaceutical PC device 9. In order to account for each new medication taken processed by the pharmaceutical account 8, each new medication request is associated to pharmacy information and a pickup date and time and the pharmaceutical account 8 is associated to contact information. The first radio communication module 7 is communicably coupled to the third radio communication module 10 in order to associate a new medication request with the patient profile. A current date and time are designated as the pickup date and time and the contact information as the pharmacy information with the remote server 1. The new medication request is then appended into the at least one taken medication for the patient profile, thereby holding the pharmaceutical account 8 liable for any possible negative reaction associated with the new medication request.

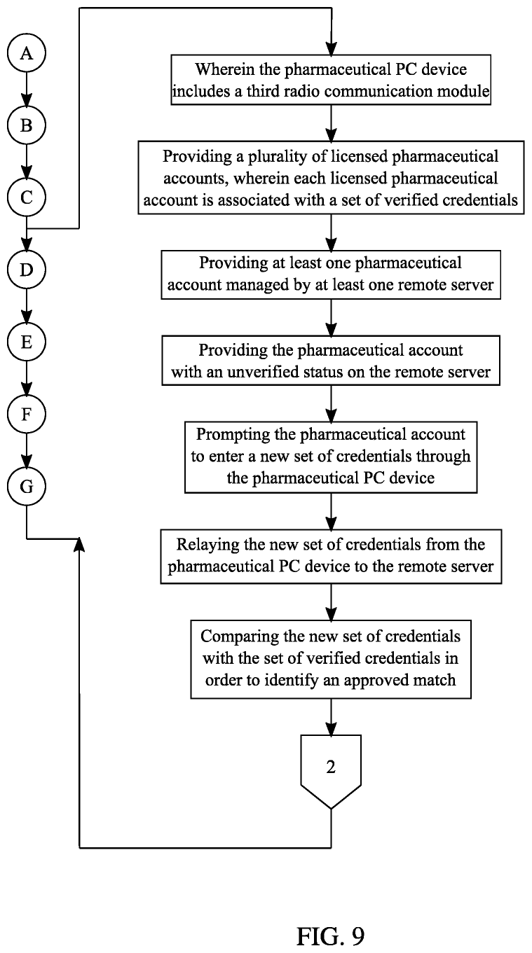

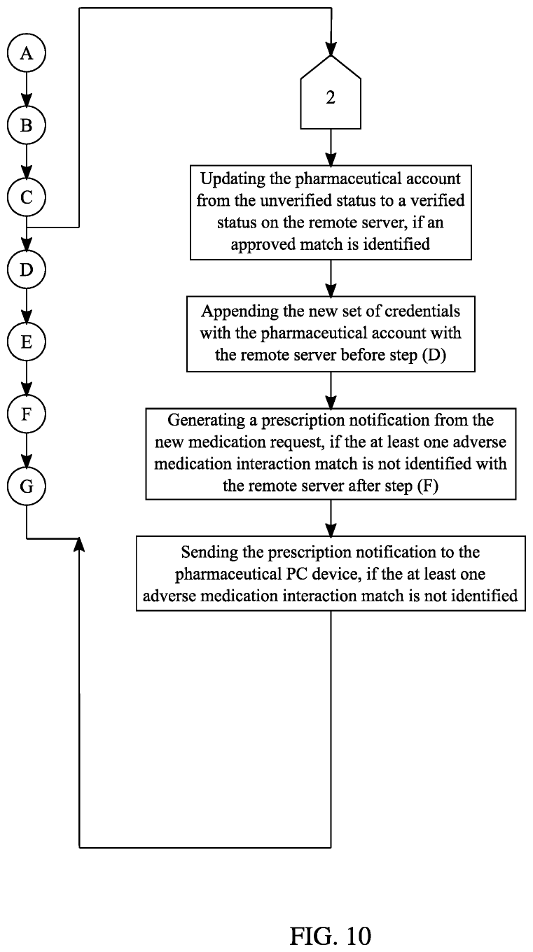

The status of the pharmaceutical account 8 is confirmed similar to the medical professional account 2 so that each patient profile is accessible by authorized accounts and the medical information of the patient account 5 is further protected, shown in FIG. 9. In order to confirm the status of the pharmaceutical account 8, a plurality of licensed pharmaceutical accounts is provided, wherein each licensed pharmaceutical account 8 is associated with a set of verified credentials. Initially, the pharmaceutical account 8 is provided with an unverified status on the remote server 1. The pharmaceutical account 8 is prompted to enter a new set of credentials through the pharmaceutical PC device 9, and the new set of credentials is relayed from the pharmaceutical PC device 9 to the remote server 1. The new set of credentials is compared with the set of verified credentials in order to identify an approved match. The pharmaceutical account 8 is able to access a patient account 5 once the pharmaceutical account 8 is updated from the unverified status to a verified status on the remote server 1, if an approved match is identified. The new set of credentials is then appended with the pharmaceutical account 8 with the remote server 1 before step (D), seen in FIG. 10.

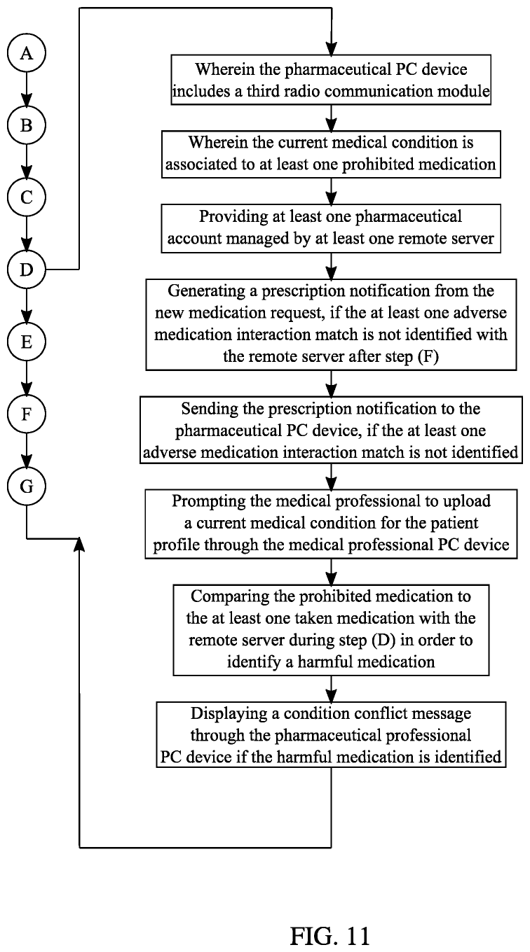

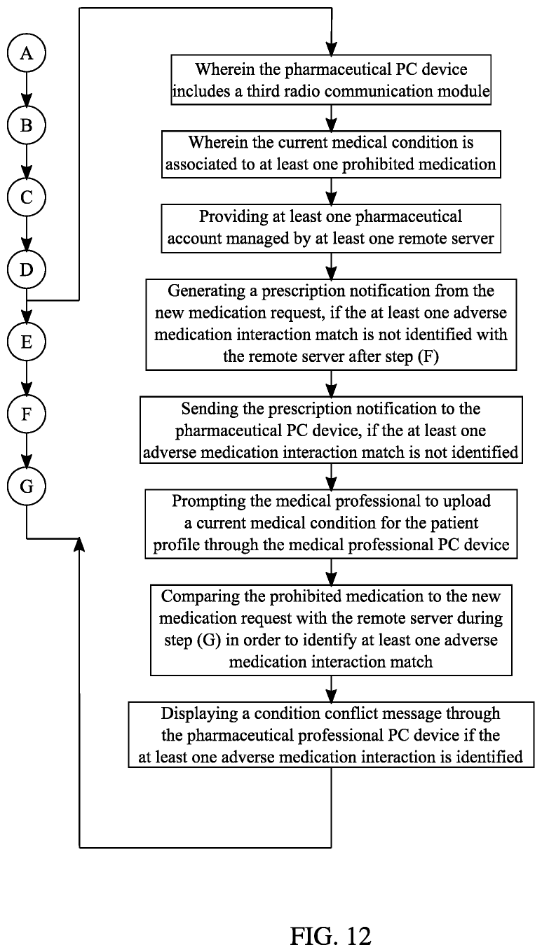

In order for the pharmaceutical account 8 to be aware of any updates of a patient profile by the medical professional account 2, a condition conflict message is displayed through the pharmaceutical professional PC device if the harmful medication is identified, seen in FIG. 11. Similarly, a condition conflict message is displayed through the pharmaceutical professional PC device if the at least one adverse medication interaction is identified, seen in FIG. 12.

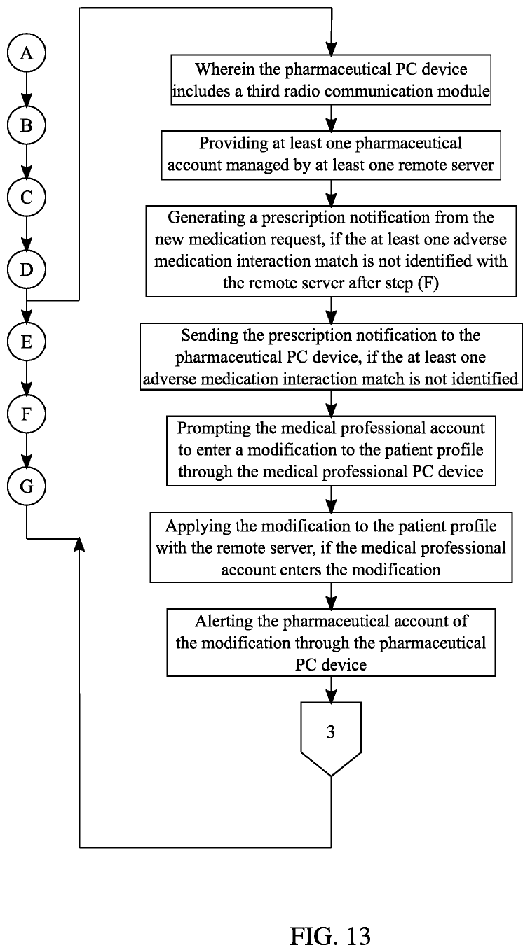

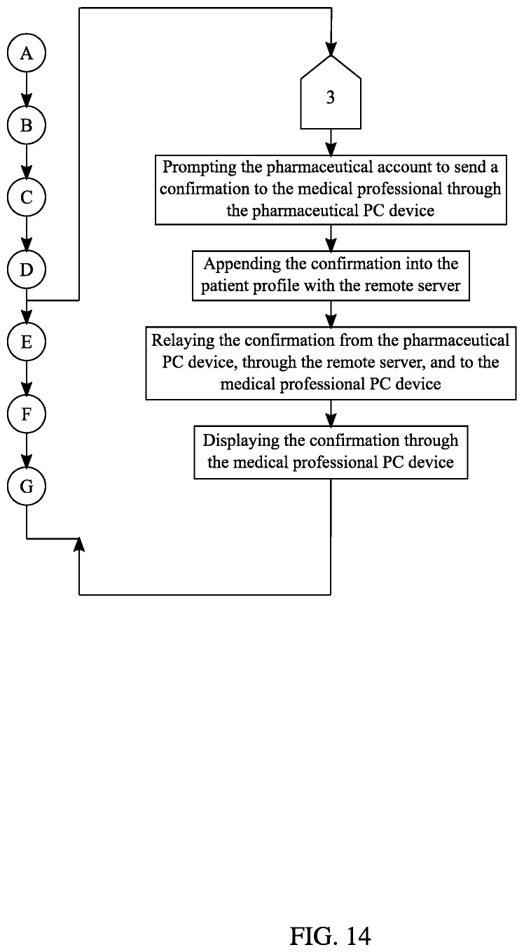

The medical professional account 2 is ensured that the pharmaceutical account 8 did not overlook any important update regarding the patient profile with the present invention. Any updates regarding the patient profile begins by prompting the medical professional account 2 to enter a modification to the patient profile through the medical professional PC device 3, seen in FIG. 13. The modification is applied to the patient profile with the remote server 1, if the medical professional account 2 enters the modification, and the pharmaceutical account 8 is alerted of the modification through the pharmaceutical PC device 9. In order to confirm the review of the modification by the pharmaceutical account 8, the pharmaceutical account 8 is prompted to send a confirmation to the medical professional through the pharmaceutical PC device 9. The confirmation is appended into the patient profile with the remote server 1, holding the pharmaceutical account 8 accountable for any action by the pharmaceutical account 8 after the modification. The confirmation is relayed from the pharmaceutical PC device 9 through the remote server 1, and to the medical professional PC device 3. The medical professional account 2 is alerted of the review by the pharmaceutical account 8 as the confirmation is then displayed through the medical professional PC device 3, seen I FIG. 14.

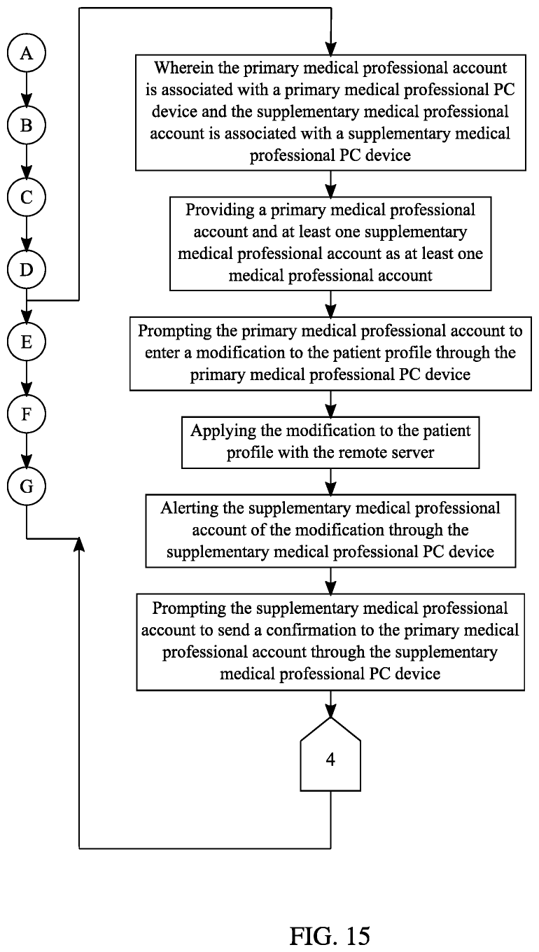

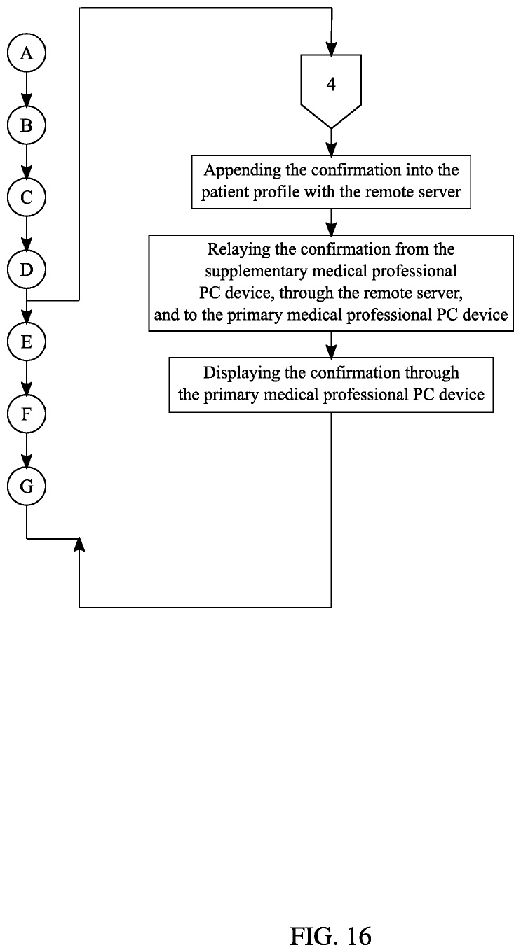

In the event the patient account 5 is monitored by more than one medical professional account 2, it essential that any and all updates by one medical professional account 2 is also reviewed by other medical professional accounts associated with the patient account 5. Seen in FIG. 15 and FIG. 16, a primary medical professional account 2 and at least one supplementary medical profession account as at least one medical professional account 2 is provided in order to differentiate multiple medical professionals associated to the patient account 5. The primary medical professional account 2 is associated with a primary medical professional PC device 3 and the supplementary medical professional account 2 is associated with a supplementary medical professional PC device 3. Similarly, the supplementary medical professional account 2 is associated with a supplementary medical professional PC device 3. The primary medical professional account 2 and the supplementary medical professional account 2 may therefore access and modify the patient account 5 independently of each other. The primary medical professional account 2 is prompted to enter a modification to the patient profile through the primary medical professional PC device 3. In order to associate modification to the patient profile, the modification is applied to the patient profile with the remote server 1. The supplementary medical professional account 2 is alerted of the modification through the supplementary medical professional PC device 3, allowing the supplementary medical professional account 2 to review all updates with the patient profile. The supplementary medical professional account 2 is then prompted to send a confirmation to the primary medical professional account 2 through the supplementary medical professional PC device 3. The confirmation is appended into the patient profile with the remote server 1, accounting for all actions of medical professional accounts associated with the patient profile. The confirmation is relayed from the supplementary medical professional PC device 3, through the remote server 1, and to the primary medical professional PC device 3. The confirmation is then displayed through the primary medical professional PC device 3 so that the primary medical professional PC account is ensured all medical professional accounts are aware of any changes to the patient profile.

Although the invention has been explained in relation to its preferred embodiment, it is to be understood that many other possible modifications and variations can be made without departing from the spirit and scope of the invention as hereinafter claimed.

What is claimed is:

1. A method of universal medical information distribution, the method comprises the steps of:
(A) providing at least one medical professional account managed by at least one remote server, wherein the at least one medical professional account is associated to a medical professional personal computing (PC) device;
(B) providing at least one patient account and plurality of medications managed by the remote server, wherein each patient account is associated with at least one taken medication from the plurality of medications, and wherein each taken medication is associated to at least one conflicting medication from the plurality of medications and, wherein each patient account is stored on a patient identifier device;
(C) prompting the medical professional account to upload a patient history through the medical professional PC device;
(D) generating a patient profile for the patient account with the remote server, wherein the patient profile includes the patient history and the taken medications;
(E) prompting the medical professional account to upload a new medication request through the medical professional PC device;
(F) comparing the new medication request to the at least one conflicting medication with the remote server in order to identify at least one adverse medication interaction match; and,
(G) displaying an adverse medication notification with the medical professional PC device if the at least one adverse medication interaction is identified.

2. The method of universal medical information distribution, the method as claimed in 1 comprises the steps of:
wherein the patient identifier device includes a first radio communication module;
wherein the medical professional PC device includes a second radio communication module;
sending the patient profile from the first radio communication module to the second radio communication module; and,
granting access to the medical professional account to view the patient profile with the medical professional PC device.

3. The method of universal medical information distribution, the method as claimed in 1 comprises the steps of:
providing a plurality of licensed medical professional accounts, wherein each licensed medical professional account is associated with a set of verified credentials;
providing the medical professional account with an unverified status on the remote server;
prompting the medical professional account to enter a new set of credentials through the medical professional PC device;
relaying the new set of credentials from the medical professional PC device to the remote server;
comparing the new set of credentials with the set of verified credentials in order to identify an approved match;
updating the medical professional account from the unverified status to a verified status on the remote server, if an approved match is identified; and,
appending the new set of credentials with the medical professional account with the remote server before step (D).

4. The method of universal medical information distribution, the method as claimed in 1 comprises the steps of:
prompting the medical professional account to upload a current medical condition for the patient profile through the medical professional PC device, wherein the current medical condition is associated to at least one prohibited medication;
comparing the prohibited medication to the new medication request with the remote server during step (G) in order to identify at least one adverse medication interaction match; and,
displaying a condition conflict message through the medical professional PC device if the at least one adverse medication interaction is identified.

5. The method of universal medical information distribution, the method as claimed in 1 comprises the steps of:
prompting the medical professional to upload a current medical condition for the patient profile through the medical professional PC device, wherein the current medical condition is associated to at least one prohibited medication;
comparing the prohibited medication to the at least one taken medication with the remote server during step (G) in order to identify a harmful medication; and,
displaying a condition conflict message through the medical professional PC device if the harmful medication is identified.

6. The method of universal medical information distribution, the method as claimed in 1 comprises the steps of:
providing at least one pharmaceutical account managed by at least one remote server, wherein the pharmaceutical account is associated to a pharmaceutical PC device;
generating a prescription notification from the new medication request, if the at least one adverse medication interaction match is not identified with the remote server after step (F); and,
sending the prescription notification to the pharmaceutical PC device, if the at least one adverse medication interaction match is not identified.

7. The method of universal medical information distribution, the method as claimed in 6 comprises the steps of:
wherein the patient identifier device includes a first radio communication module;
wherein the pharmaceutical PC device includes a third radio communication module;
sending the patient profile from the first radio communication module to the third radio communication module; and,
granting access to the pharmaceutical account to view the patient profile with the pharmaceutical PC device.

8. The method of universal medical information distribution, the method as claimed in 7 comprises the steps of:
wherein each new medication request is associated to pharmacy information and a pickup date and time;
wherein the pharmaceutical account is associated to contact information;
communicably coupling the first radio communication module to the third radio communication module;
designating a current date and time as the pickup date and time and the contact information as the pharmacy information with the remote server; and,
appending the new medication request into the at least one taken medication for the patient profile.

9. The method of universal medical information distribution, the method as claimed in 6 comprises the steps of:
providing a plurality of licensed pharmaceutical accounts, wherein each licensed pharmaceutical account is associated with a set of verified credentials;
providing the pharmaceutical account with an unverified status on the remote server;
prompting the pharmaceutical account to enter a new set of credentials through the pharmaceutical PC device;
relaying the new set of credentials from the pharmaceutical PC device to the remote server;
comparing the new set of credentials with the set of verified credentials in order to identify an approved match;
updating the pharmaceutical account from the unverified status to a verified status on the remote server, if an approved match is identified; and,
appending the new set of credentials with the pharmaceutical account with the remote server before step (D).

10. The method of universal medical information distribution, the method as claimed in 6 comprises the steps of:
prompting the medical professional to upload a current medical condition for the patient profile through the medical professional PC device, wherein the current medical condition is associated to at least one prohibited medication;
comparing the prohibited medication to the at least one taken medication with the remote server during step (D) in order to identify a harmful medication; and,
displaying a condition conflict message through the pharmaceutical professional PC device if the harmful medication is identified.

11. The method of universal medical information distribution, the method as claimed in 6 comprises the steps of:
prompting the medical professional to upload a current medical condition for the patient profile through the medical professional PC device, wherein the current medical condition is associated to at least one prohibited medication;
comparing the prohibited medication to the new medication request with the remote server during step (G) in order to identify at least one adverse medication interaction match; and,
displaying a condition conflict message through the pharmaceutical professional PC device if the at least one adverse medication interaction is identified.

12. The method of universal medical information distribution, the method as claimed in 6 comprises the steps of:
prompting the medical professional account to enter a modification to the patient profile through the medical professional PC device;
applying the modification to the patient profile with the remote server, if the medical professional account enters the modification;
alerting the pharmaceutical account of the modification through the pharmaceutical PC device;
prompting the pharmaceutical account to send a confirmation to the medical professional through the pharmaceutical PC device;
appending the confirmation into the patient profile with the remote server;
relaying the confirmation from the pharmaceutical PC device, through the remote server, and to the medical professional PC device; and,
displaying the confirmation through the medical professional PC device.

13. The method of universal medical information distribution, the method as claimed in 1 comprises the steps of:
providing a primary medical professional account and at least one supplementary medical professional account as at least one medical professional account, wherein the primary medical professional account is associated with a primary medical professional PC device and the supplementary medical professional account is associated with a supplementary medical professional PC device;
prompting the primary medical professional account to enter a modification to the patient profile through the primary medical professional PC device;
applying the modification to the patient profile with the remote server;
alerting the supplementary medical professional account of the modification through the supplementary medical professional PC device;
prompting the supplementary medical professional account to send a confirmation to the primary medical professional account through the supplementary medical professional PC device;
appending the confirmation into the patient profile with the remote server;
relaying the confirmation from the supplementary medical professional PC device, through the remote server, and to the primary medical professional PC device; and,
displaying the confirmation through the primary medical professional PC device.
